(19) United States
(12) Patent Application Publication
(10) Pub. No.: US 2018/0122500 A1
(43) Pub. Date: May 3, 2018

(54) SYSTEMS AND METHODS PROVIDING CENTRALIZED MEDICAL PRIVILEGING AND DATA

(71) Applicant: Jennifer Harrell Vines, Dallas, TX (US)

(72) Inventor: Jennifer Harrell Vines, Dallas, TX (US)

(21) Appl. No.: 15/725,833

(22) Filed: Oct. 5, 2017

Related U.S. Application Data
(60) Provisional application No. 62/404,560, filed on Oct. 5, 2016.

(51) Int. Cl. G16H 10/60; G06F 21/62; H04L 29/06

(52) U.S. Cl. CPC G16H 10/60; H04L 63/08; G06F 21/6263

(57) ABSTRACT

Systems and methods provide credentialing and privilege processing, management, control, and exchanges of data for authorized users. A server accessible from clients over a data network can provide centralized credentialing forms, receive supplemental information in association with medical provider application, and authenticate and grant access to records and forms associated with the medical provider to hospitals and third party institutions authorized by the medical provider to access and provide records in support of the medical provider's application for privileges, and thereby access, to medical providing institutions.

### CROSS REFERENCE TO RELATED APPLICATIONS

This patent application claims the priority and benefit under 35 U.S.C. § 119(e) of U.S. Provisional Patent Application Ser. No. 62/404560, filed on Oct. 5, 2016, and entitled “SYSTEMS AND METHODS PROVIDING CENTRALIZED MEDICAL PRIVILEGING AND DATA,” which is incorporated herein by reference in its entirety.

### FIELD OF THE EMBODIMENTS

The embodiments are generally related to medical provider credentialing hospital privileging. More particularly the embodiments are related to systems and methods centralizing and streamlining hospital privileging for medical providers, which can decrease the time required to apply for privileges and reduce administrative costs for hospitals.

### BACKGROUND

The U.S. Department of Health and Human Services' definition of credentialing “the process of assessing and confirming the qualifications of a licensed or certified health care practitioner.” Credentialing is an, essential process that healthcare organizations and practices must perform to ensure those providing services are qualified to do so.

The process has become more complicated over time due to the expansion of providers' scopes of service, the requirements of third-party payers (e.g., the U.S. government and private health insurance plans), and organizational standards (accrediting bodies).

The companion piece to credentialing is “privileging,” which is the process of authorizing a licensed or certified healthcare practitioners specific scope of patient care services. Privileging is performed in conjunction with an evaluation of an individual's clinical qualifications and/or performance.

In the past, credentialing and privileging were mainly associated with hospitals. Now, these processes are being required at healthcare facilities, ambulatory surgical centers, and long-term care organizations.

Healthcare organizations are responsible for validating the competency of their medical staff through credentialing and privileging. These processes are closely tied to reimbursement, accreditation standards, and state and federal laws. Although credentials and privileges will vary among providers (depending on their backgrounds, qualifications, areas of practice, and practice settings), having detailed and consistent credentialing and privileging processes is imperative.

The hospital privileging process is dysfunctional and extremely time consuming. Many hospitals continue to use paper applications while constantly receiving incomplete submission files that delay their ability to hire a provider. Currently, medical professionals who are applying for privileging at a hospital must print multiple applications and forms to be completed and returned. In addition, all medical school and licensing verification is also completed via paper forms. All of the paper documents are either saved in physical file folders or scanned and saved to management software. The process is scattered and time consuming.

The client health system has recognized that the manner in which its credentialing activities are currently being carried out across its system does not consistently achieve the level of quality, timeliness, efficiency, high reliability, and value needed to succeed in today's environment. Specific challenges include: inconsistency and lack of standardization with credentialing and privileging processes; lengthy turnaround times for the initial appointment process; and lack of physician satisfaction with the credentialing and privileging process.

Centralized credentialing can resolve some of the complexities of credentialing and privileging, however, the unique nuances of each hospital's bylaws and how they impact the delineation of privileges seems to be a barrier to such a product/service existing up to now (as the bylaws would need to be standardized across participating hospitals). What is needed are systems and methods to streamline the hospital privileging process, decreases the time required to apply for privileges, and reduces administrative costs for hospitals.

### SUMMARY OF THE EMBODIMENTS

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

The present inventor hereby discloses many features believed to overcome the current problems associated with professional credentialing and privilege (health care institution access) management. It is a feature of the embodiments to provide a centralized application for the hospital privileging process.

In accordance with features of the embodiments, with a centralized system users can remotely access servers containing credentialing and managing privileges for health care providers using their electronic clients (e.g., desktops computers, tablets, smartphones). In accordance with an example embodiment, a network accessible server is provided that is configured to register users, collect data, process data, authorize access to the exchange or provision of data to requesting users and essentially streamline the privilege management process.

For hospitals, features of the embodiments can enable hospitals to:
Eliminate paper applications and forms
Retrieve and download application files of medical professionals
Efficiently utilize centralized credentialing and privileging systems for initial and renewal privileging
First source verify references
Gain easy access licensing documentation.

For medical professionals, features of the embodiments can enable them to:
Enter application information once, which can be saved for future use
Eliminate the need for repetitive record retrieval from former employers, licensing agencies, medical schools
Enable worldwide access to data
Enable single contact and verification of references, with reference information being saved in a medical provider profile.
Provide an economically feasible profile management and access from one centralized platform.

Further embodiments can provide a link to be placed on a hospital's website that can direct users (e.g., a medical professional) to a secure website and server.

It is another feature that, during registration, a medical professional can initially set up a user account and profile including completing ONE application that is a compilation of numerous application fields from a variety of hospitals. The application can be saved to the professional's profile in a remote server (e.g., the service cloud). In addition, the professional can request references from others either by searching and locating someone who is currently using the service, or by sending an email that allows them access to the service. The references will complete ONE reference form and that will be saved to the professional's profile. The professional will also have the ability to upload medical school documentation, licensing verification, or certification verification. These documents will he saved to the professional's profile. When the profile is complete, professionals can search for hospitals to select in receiving their privileging application. The particular hospital's privileging fee will be charged via the service provider, along with a nominal fee per application. Furthermore, medical professionals will have the opportunity to maintain their privileges profile for a monthly maintenance fee. This information will remain accessible to them while allowing for additional information to be uploaded. Future privileging applications can be submitted via the service.

In accordance with features of the present embodiments, hospitals can more efficiently process new and renewal privileging applications via the system. Hospitals can have user credentials with which to access a dashboard. This dashboard can provide a page on which to choose fields from the general application that meet their needs. Hospitals will be alerted to privileging applications for their institution and can view the medical professional's profile, application, and documents. The hospital can then have the ability to categorize each applicant based on the action taken. Hospitals can choose whether to maintain their applicant information in the service cloud or download it to their information management systems.

In accordance with features of the present embodiments, references can be requested via the medical professional, but an additional level of security can be provided to ensure first source verification. References can utilize a link to access their dashboard, and once a profile is created, a verification code can be emailed. This code can be required to log-in and continue to the reference form. The referral form can be completed once and electronically signed, then uploaded to the medical professional's profile.

In accordance with additional features of the embodiments, time saving features enable healthcare providers to complete and attest to multiple state credentialing applications in one intelligent workflow design, upload supporting documents directly into a database to eliminate the need for manual submission, and to improve the timeliness of completed applications, review and approve information before data is imported, protect against delays in data processing with more focused prompts and real-time validation, and enable self-registration with the system before a health plan initiates the application process.

### BRIEF DESCRIPTION OF THE DRAWING

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a block diagram of a system that can be used o it implement embodiments of the present invention;

FIG. 2 illustrates a flow diagram for a process in accordance with the embodiments;

FIG. 3 illustrates a flow diagram for a process in accordance with he embodiments; and

FIG. 4 illustrates a flow diagram for a process in accordance with the embodiments.

### DETAILED DESCRIPTION

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof (other than software per se). The following detailed description is therefore not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment, and the phrase “in another embodiment” as used herein does not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms, such as “a,” “an,” or “the,” again, may be understood to convey a singular usage or to convey a plural usage, depending at least in part upon context. In addition, the term “abased on” may be understood as not necessarily intended to convey an exclusive set of factors and may, instead, allow for existence of additional factors not necessarily expressly described, again, depending at least in part on context.

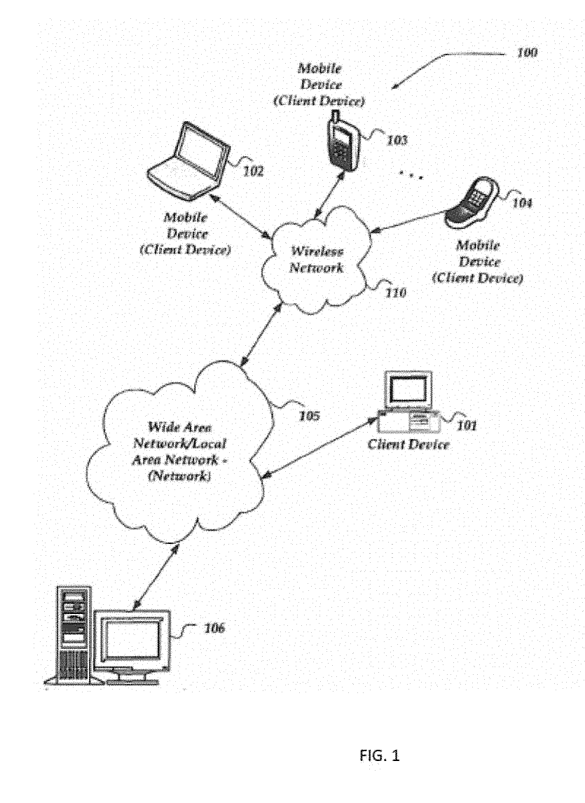

FIG. 1 illustrates a schematic diagram depicting an example embodiment of a system 100 composed of one or more networks. System 100 is an example of an environment in which a system for mapping notable entities to their social profiles, as described in greater detail herein, may operate. Other embodiments that may vary, for example, in terms of arrangement or in terms of type of components are also intended to be included within the claimed subject matter. The system 100 depicted in FIG. 1, for example, can include a variety of networks, such as a WAN (Wide Area Network)/LAN (Local Area Network) 105, a wireless network 110, and a variety of devices, such as a client device 101 and mobile devices 102, 103, 104, and a variety of servers, such as credentialing and privileges server (SAS) 106. Server 106 can also be representative cloud-based services. In the example configuration depicted in FIG. 1, mobile devices 102, 103, and 104 are client devices that communicate wirelessly with system 100 through the wireless network 110. The WAN/LAN network 105 also communicates with the wireless network 110.

A server, such as credentialing/privilege server 106, may include a device that includes a configuration to receive, process, and provide medical professional information and related data via a network to another device. A SAS server 106 may, for example, host a website, and can enable secure log-in functionality for users to engage with and provide/receive data from the server 106.

A network such as network 105 and/or network 110 depicted in FIG. 1 can couple devices so that communications and data may be exchanged, such as between a server 106 and a client device 101 or other types of devices, including between wireless devices 103/104 coupled via a wired or wireless network 110, for example. A network may also include mass storage, such as network-attached storage (NAS), a storage area network (SAN), or other forms of computer or machine-readable media, for example. A network may include the Internet, one or more Local Area Networks (LANs), one or more Wide Area Networks (WANs), wire-line type connections, wireless type connections, or any combination thereof. Likewise, sub-networks may employ differing architectures or may be compliant or compatible with differing protocols, and may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices can be remotely coupled to a network, such as via a telephone line or link, for example.

A wireless network such as the wireless network 110 depicted in FIG. 1 may couple client devices with the network. That is, such a wireless network may employ stand-alone ad-hoc networks, mesh networks, wireless LAN (WLAN) networks, cellular networks, or the like. A wireless network such as wireless network 110 can further include a system of terminals, gateways, routers, or the like coupled by wireless radio links, or the like, which may move freely, randomly, or organize themselves arbitrarily, such that network topology may change, at times even rapidly. A wireless network may further employ a plurality of network access technologies, including Long Term Evolution (LTE), WLAN, Wireless Router (WR) mesh, or 2nd, 3rd, or 4th generation (2G, 3G, 4G, 5G, and so on) cellular technology, or the like. Network access technologies may enable wide area coverage for devices, such as client devices with varying degrees of mobility, for example.

For example, a network may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

Signal packets communicated via a network, such as a network of participating digital communication networks (e.g., networks 105, 110) may be compatible with or compliant with one or more protocols. The signaling formats or protocols employed may include, for example, TCP/IP, UDP, DECnet, NetBEUI, IPX, AppleTalk, or the like. Versions of the Internet Protocol (IP) may include IPv4 or IPv6.

The Internet refers to a decentralized global network of networks. The Internet can include Local Area Networks (LANs), Wide Area Networks (WANs), wireless networks, or long haul public networks that, for example, allow signal packets to be communicated between LANs. Signal packets may be communicated between nodes of a network, such as, for example, to one or more sites employing a local network address. A signal packet may, for example, be communicated over the Internet from a user site via an access node coupled to the Internet. Likewise, a signal packet may be forwarded via network nodes to a target site coupled to the network via a network access node, for example. A signal packet communicated via the Internet may, for example, be routed via a path of gateways, servers, etc., that may route the signal packet in accordance with a target address and availability of a network path to the target address.

A website in accordance with embodiment of the present invention can function in or with credentialing/privilege server 106 and allow for three separate user types to log-in: hospitals (both current and prospective), medical service providers/professionals (e.g., Medical Doctors), and third parties associated with hospitals/professionals (e.g., insurance carriers, etc.).

For hospitals, features of the embodiments can enable hospitals to:
Eliminate paper applications and forms
Retrieve and download application foes of medical professionals
Efficiently utilize centralized credentialing and privileging systems for initial and renewal privileging
First source verify references
Gain easy access licensing documentation.

For medical professionals, features of the embodiments can enable them to:
Enter application information once, which can be saved for future use
Eliminate the need for repetitive record retrieval from former employers, licensing agencies, medical schools.
Enable worldwide access to data
Enable single contact and verification of references, with reference information being saved in a medical provider profile.
Provide an economically feasible profile management and access from one centralized platform.

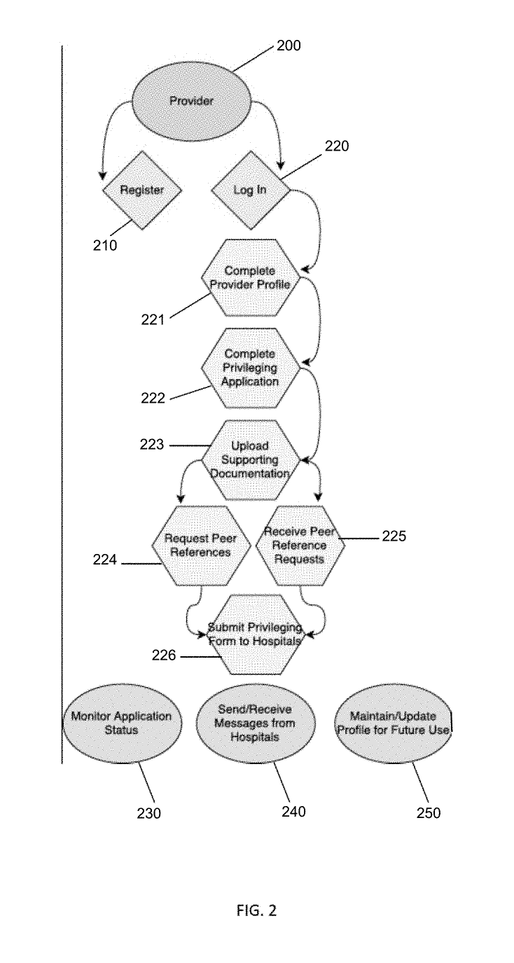

Referring to FIG. 2, a flow diagram of example modules a system in accordance with features of the embodiments is illustrated. A centralized credentialing and privilege management/administration system can support the delivery of electronic documents to authorized (e.g., registered) users, and eliminate the need for paper application in the credentialing and privileging process. A medical provider 200 (“Provider”) can register with a centralized credentialing and privilege management system as shown at the diamond block 210 entitled “Register.” Registration can include setting up a personal profile, filing out an application, uploading credential documents (e.g., degrees, licenses), and identifying references. After being registered, the Provider can log-in to the system as shown in the block 220 labeled “Log in.” When logged into the system, a Provider can: Complete Provider Profile 221, Complete Privileging Application 222, Upload Supporting Documentation 223, Request Peer References 224, Receive Peer References Requests 225, and Submit Privileging Form to Hospitals 226. Once the Provider has a completed credentialing package stored in a database within a remote server (e.g., in the cloud), the Provider can Monitor Application Status 230, Send/Receive Messages from Hospitals 240, and Maintain/Update a Profile for Future Use 250.

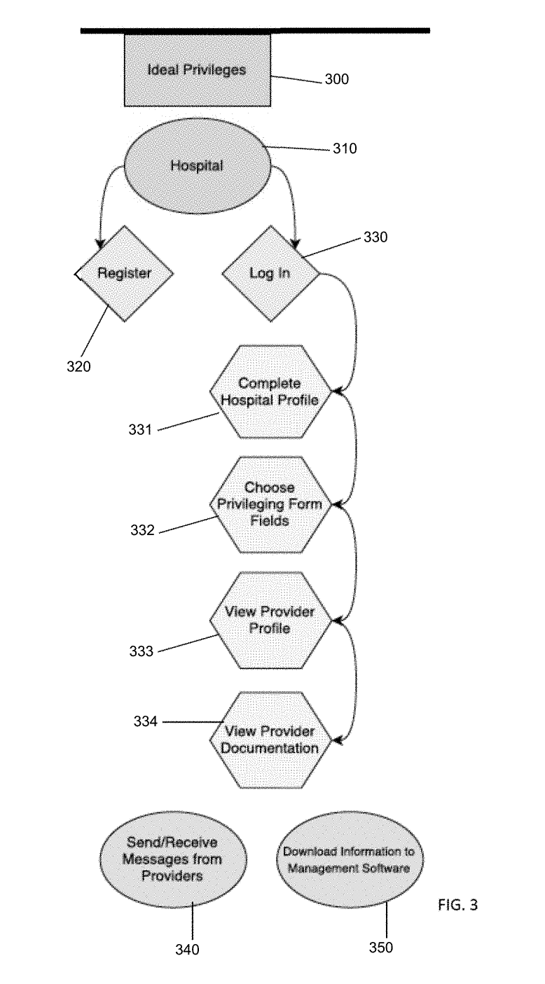

Referring to the flow diagram in FIG. 3, an application 300 referred to as “Ideal Privileges” can provide a healthcare institution 310 (e.g., “Hospital”) access to the credentialing and privileging service system. A hospital (like the healthcare provider) can register 320 with the system. Once registered, the hospital can log-in to the system as shown at block 330. Once logged in, the hospital can complete Hospital Profiles 331, Choose Privileging Form Fields 332, view Provider Profiles 333, and view Provider Documentation 334. Once registered and obtaining access to data, hospitals can Send/Receive message from Providers 340 and Download Information to Management Software 350.

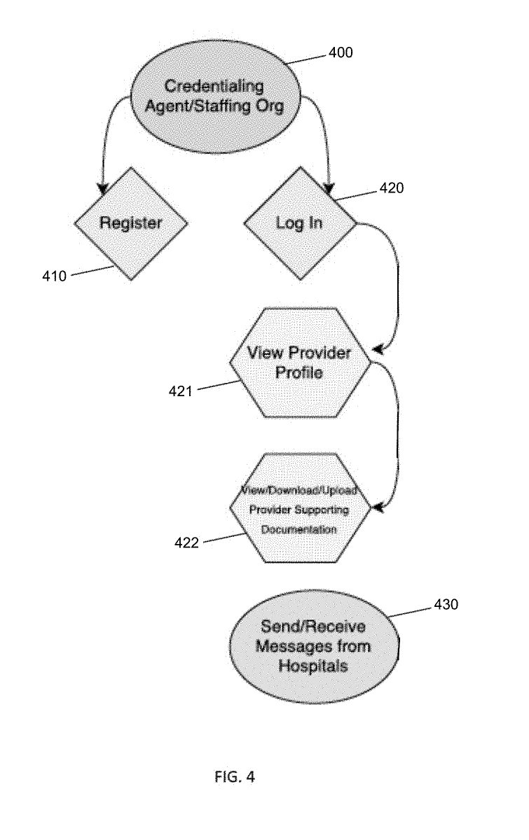

Referring to the flow diagram in FIG. 4, third parties 400 (e.g., “Credentialing Agent/Staffing Org”) can access the credentialing and privileging management system. Like the provider and hospitals, the credentialing agents can register 410 with the system and log-in 420 to the system to obtain access to data. The third parties can View Provider Profiles 421 and View and Download/Upload Provider Supporting Documentation 422. The third parties can also send/receive messages from hospitals and service providers 430.

1. A system for providing credentialing and privilege processing, management, control, and exchanges of data for authorized users, comprising:
a server accessible from clients over a data network, the server providing a centralized credentialing forms, receiving supplemental information in association with a medical provider applications, and authenticating and granting access to records and forms associated with the medical provider to hospitals and third party institutions authorized by the medical provider to access and provide records in support of the medical provider's application for privileges, and thereby access, to medical providing institutions.

2. A method for providing centralized credentialing and privilege management, processing, control, and exchanges for authorized users, comprising:
registering medical professionals, healthcare institutions, and affiliated third party users with access to a remote, secured server; and
enabling a medical professional to grant healthcare institutions and affiliated third party users access to data associated with the medical professional in support of the medical professional's application for privileges, and thereby access, to healthcare institutions.
